(19) United States
(12) Patent Application Publication
(10) Pub. No.: US 2009/0239302 A1
(43) Pub. Date: Sep. 24, 2009

(54) Method for Constructing Functional Living Materials, Resulting Materials and Uses Thereof

(76) Inventors: Gero Decher, Kehl-marlen (DE); Olivier Felix, Holtzheim (FR); Benjamin Saulnier, Nantes (FR); Albert Izquierdo, Versailles (FR); Jean-Claude Voegel, Valff (FR); Pierre Gilbert Schaaf, Molsheim (FR); Nadia Jessel, Keinheim (FR); Vincent Paul Ball, Marienthal (FR)

Correspondence Address: NIXON & VANDERHYE, PC, 901 NORTH GLEBE ROAD, 11TH FLOOR, ARLINGTON, VA, 22203, US

(21) Appl. No.: 12/227,216

(22) PCT Filed: May 16, 2007
(86) PCT No.: PCT/FR2007/000836

§ 371 Date: Mar. 9, 2009

(30) Foreign Application Priority Data
May 16, 2006 (FR) 06/04358

(51) Int. Cl. C12N 5/06

(52) U.S. Cl. 435/402; 435/395

(57) ABSTRACT

The invention concerns a method for constructing a functional living biomaterial, characterized in that it consists in assembling layer by layer (3D) a matrix and 2D layers of functional living cells by controlling their interactions and 3D structuring based on the desired organization and final shape of the biomaterial, wherein each layer has its own pattern adapted to the neighbouring layers and on the functionality of whole synthetic biomaterials. The invention is useful in the field of biomedicine and nanobiotechnology.

The object of this invention is a method for constructing functional biomaterials. This invention also relates to biomaterials obtained using this method, and to applications thereof. Herein, the term “biomaterial” is intended to refer to a 3D object (biohybrid) made up, as a minimum, of macromolecules, living cells, and other components (molecules, particles, vesicles, etc.) such as, for example, a tissue or an organ, unless otherwise indicated.

It is known that a strong demand exists for functional biomaterials, for the repair of tissues that have been destroyed or damaged by diseases, and in particular a crucial need exists for organs for transplants.

Various methods have been proposed to meet these needs, but these are nowhere near allowing routine production to take place, even of simple tissues.

The conventional method used in tissue engineering involves the colonisation of a porous inorganic or hybrid matrix by cells (1). However, this method does not allow complex biomaterials—which rely on the organisation of both a large number of cells and of different cells—to be created, since there is no control over the structure of the tissue generated and of the individual environment around each cell type.

A method for transferring a cell layer onto a cell support has also been proposed (2) and applied to the stacking of a few cell layers. However, this method does not allow any control over the 2D and 3D structure of a biomaterial and the individual environment around each cell.

According to two more recent methods, the present authors have proposed the deposition of viable cells onto a surface using a living cell dispenser (cell print), or preferably using an ink-jet printer (3) or through laser printing (4). A 3D organisation is obtained through the successive deposition of a support layer made up of a hydrogel, a layer of cells, and another support layer. However, the biomaterial that is obtained does not provide the characteristics that a 3D biohybrid requires to be used as a tissue, and even less so as a functional organ. The use of a hydrogel as an extracellular matrix does not allow control over the 2D and 3D organisation of a biomaterial and the individual environment around each cell

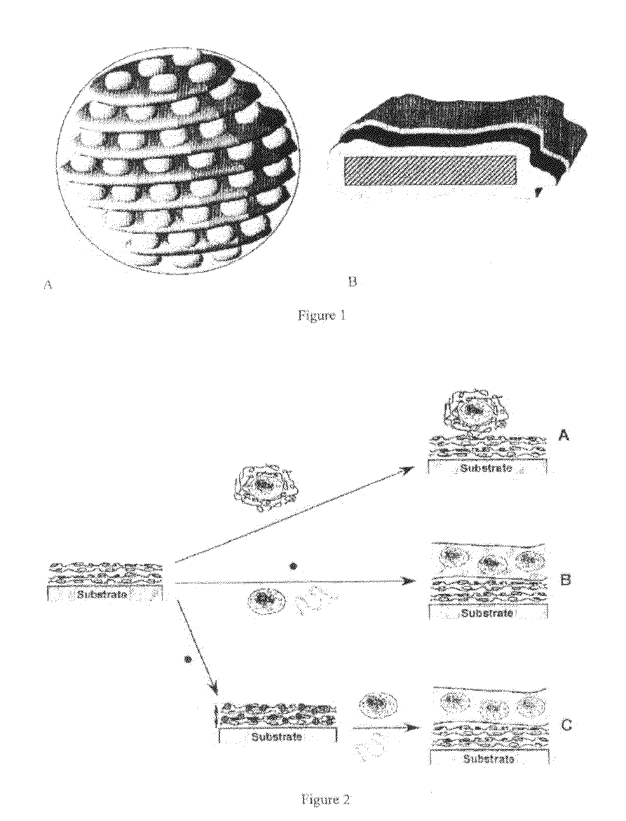

Research by the inventors in this field has led them to develop means—by using a deposition method known as “layer-by-layer” (abbreviated to “LbL”) (5-10)—to achieve an organisation of all the components required to obtain a functional living biomaterial, in particular to construct a functional tissue or organ (complete biohybrid), as well as a functional biohybrid structure, around an implant or an implantable biomedical device (partial biohybrid) (FIG. 1).

It is therefore an object of the invention to provide a method for constructing functional living biomaterials that meet the needs of the art in this field and may be routinely used due to their ease of use and low manufacturing cost.

The invention also relates to biomaterials obtained using this method as new products, and to their applications, in particular in biomedical and nanobiotechnology applications.

The method of construction of an artificial functional living biomaterial according to the invention is characterised by the layer-by-layer assembly (3D) of a matrix and of layers (2D) of functional living cells by controlling their interactions and the 3D structure depending on the final desired organisation and form of the biomaterial (tissue or organ), in which each layer has its own pattern which is suited to the neighbouring layers and to the functionality of the entire artificial biomaterials.

More particularly, 2D cell layers are formed either in a homogenous manner (non-structured layers) by soaking, spraying (11) or using a method which gives a thin hydrogel layer (with nanometre to micrometre-scale thickness) during the immobilisation period, or in a heterogeneous manner (layers structured in specific patterns) by cell-by-cell spotting using a printer or a robot system.

More specifically, the method according to the invention is characterised in that 2D patterns of functional living cells or stem cells, elements which are necessary for them to survive, grow and differentiate, and an extracellular matrix with a 2D and 3D structure which depends on the desired final organisation and form of the biohybrid object (tissue or organ) are successively deposited onto a support, in which each layer has its own pattern which is suited to the neighbouring layers and to the functionality of the entire artificial biomaterials.

These arrangements allow a fine three-dimensional structure of the various types of living cells and macromolecules to be created and controlled, with a precision of the order of one micrometre (2D) and one nanometre (3D), within the same biomaterial, and allows the composition of the extracellular matrix to be controlled, which is essential for the cells to survive and to communicate.

During the 2D cell-by-cell spotting in each layer and during the sequential 3D deposition of several single-cell layers and matrix layers in accordance with the layer-by-layer method, each individual cell is actually provided with, at a local level, the elements required for it to survive, such as nutrients, oxygen-transportation or scavenging agents, growth factors, cell-differentiation factors, antioxidants, cell-adhesion promoters, antiinflammatory agents, antibacterial agents, antiviral agents, angiogenesis factors or inhibitors, immunosuppressive agents, co-factors, vitamins, DNAs, gene-transfection agents or any factor which stimulates or suppresses cellular, biological or therapeutic activity.

The invention thus provides means for providing each cell with the optimum biochemical environment in relation to its needs and to its position in the biomaterial that is being created.

According to one embodiment of the invention, more specifically for the construction of a simple biomaterial, a single layer of cells is deposited, involving cells of the same type of at least two different cell types.

According to another embodiment of the invention, for the construction of a complex biomaterial, several successive alternating layers of polymers and cells of different types, depending on the desired architecture, are deposited.

The deposited cells are, for example, cells which form the final surface of the organ (skin), endothelial cells, smooth-muscle cells, connective-tissue cells, cells which make up blood vessels, cells which make up tissues or organs, and stem cells whose differentiation into functional cells is induced by differentiation factors.

Cells are deposited in accordance with the required pattern, depending on their function.

To control cell-matrix interactions, naked cells or cells which have been individually coated with polyelectrolyte multilayers (12-15), or cells which have been immobilised during surface gelification of a thin hydrogel layer (with nanometre to micrometre-scale thickness) made up, for example, of calcium alginate, are used. These various immobilisation methods make it possible to ensure attachment of cells, either in an heterogeneous manner in accordance with the patterns imposed by the deposition method and the distance between the cells (naked cells, encapsulated cells, and thin hydrogel layer), or in an homogenous manner (naked cells, encapsulated cells, and thin hydrogel layer), and to ensure the survival and maximum functionality of the cells in the biohybrid objects. The biohybrids are constructed with a 2D/3D structure in which each individual cell enjoys an optimum biochemical environment in relation to its requirements and to its position in the artificial organ.

According to an alternative embodiment of the invention, living cells are used (12-15) which are encapsulated in a coating having a nanometre-scale thickness. Suitable compounds for making the coating include functional macromolecules suitable for the coating of cells. Such encapsulation, by modifying the surface of cells, allows cells which are normally non-adherent to be used. It also allows the cell to be made “invisible” in relation to any recognition, allows entry/exit of molecules or macromolecules at the cell-environment interface to be controlled (filtration effect, selective filtration), and limits fibrosis.

The construction method defined above also includes the deposition of elements required for the survival, growth and differentiation of the cells.

Such elements include in particular, as indicated above, growth factors, antiinflammatory agents, antibacterial agents, differentiation factors or vascularisation promoters.

As regards the extracellular matrix, it is constructed either using the LbL method, from molecules, macromolecules or colloidal compounds (e.g. hydrogel in the form of nanoparticles, vesicles, etc.) which interact by covalent and/or non-covalent interactions, or by gelification of two or more compounds, such as, for example, sodium alginate and calcium. During construction, molecules may be incorporated either into the multilayer matrix or on either side of the hydrogel matrix which is forming on the surface and which is made up, for example, of calcium alginate. The matrix has a composition which is appropriate to the cell type, through the use of polymers with well-controlled chemical functions.

Advantageously, it is mainly made up of biotolerant or bio-inert macromolecules, or where appropriate, biodegradable or bioactive macromolecules.

In carrying out this invention, the immobilisation of cells onto the multilayer matrix or in the hydrogel matrix which is forming on the surface, is contemplated in accordance with 4 different methods, depending on requirements (FIGS. 2A-2D):
the first method (2A) involves the layer-by-layer deposition of naked cells (controlled cell adhesion) onto a substrate which has been modified beforehand using a multilayer treatment. The cells may then be covered using another multilayer system;
the second method (2B) involves the layer-by-layer deposition of individually encapsulated cells (4) using multilayers of polyelectrolytes, onto a substrate which has been modified beforehand using a multilayer treatment;
the third method (2C) is based on the incorporation of cells into a hydrogel matrix which is forming on the surface and whose thickness depends on the thickness of the multilayer reservoir which controls the concentration of one of the compounds necessary for gelification, such as, for example, calcium, PLL, or oligoamines. For example, the calcium alginate matrix is formed by gelification of a sodium alginate solution containing cells in contact will a gelling agent, calcium;
the fourth method (2D), as an alternative to the third method, also relies on the inclusion of cells into a hydrogel matrix which is forming on the surface and which is made up, for example, of calcium alginate, but without a multilayer reservoir being involved. The construction of the calcium alginate matrix is achieved, e.g. by simultaneous deposition of a sodium alginate solution containing cells and a calcium solution.

The nascent hydrogel matrix at the surface, obtained by spraying, may be deposited between multilayers of polyelectrolytes which contain active agents, such as, for example, growth factors and differentiation factors. This cell-immobilisation method may also be used in the construction of a biohybrid object involving a patterned structure by the deposition of gelling agents using a printer or a robot system.

The deposition of the various components involved in the construction of the biomaterial is carried out either by soaking or spraying in order to form a 2D non-structured layer, or by using a printer (FIG. 3) or a robot system in order to construct a 2D layer which is either structured in specific patterns or non-structured. The method using spraying (9, 10) has the advantage of allowing rapid and homogeneous deposition onto the surface, and also of providing totally tailor-made matrices by modulating the components throughout construction.

Biomaterials as obtained by the method defined above are new products and, as such, are therefore an object of this invention.

These biomaterials provide a solution to the acute problem of unsatisfied demand for organs for transplants which are essential for the survival of patients (total or partial biohybrids). They also offer an alternative when transplants of certain organs have a low probability of being carried out successfully. Furthermore, advantageously, their use means that high doses of immunosuppressants administered to transplant patients and which prevent them from regaining the best possible quality of life after transplantation can be done away with.

Since the structure of biomaterials according to the invention approaches that of the actual tissue, they can be used, in general, for tissue repairs, e.g. in cartilage reconstruction. In this case, hyaluronic acid, a key constituent of cartilage, is incorporated into the matrices intended for osteochondral repair. Mimicking the heterogeneous nature of native hyaline tissue, the matrices used may also include chondrocytes.

These biomaterials are therefore particularly suitable for biomedical and nanobiotechnology applications, as interface or membrane reactors as thin layers, for the biomanufacture of molecules of interest using living cells, or as artificial organs, either as complete biohybrids or as synthetic devices coated with a biohybrid layer.

Other characteristics and advantages of the invention will be given below in the description of embodiments, which must in no way be interpreted as a limitation of the invention to these characteristics.

In the following description, reference will be made to FIGS. 1-7, in which, respectively:

FIG. 1 is a schematic representation of a 2D/3D biohybrid fully constructed layer by layer (1A), and of a biohybrid constructed around an implant or an implantable biomedical device (1B);

FIG. 2 is a schematic representation of the four cell-immobilisation methods (2A-2D) used in to carry out the invention;

FIG. 3 shows a method for assembling the components of a biomaterial using an ink-jet printer;

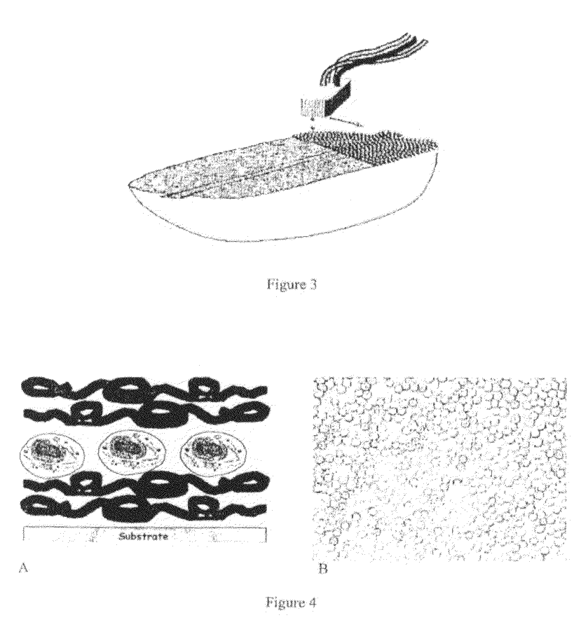

FIG. 4 shows a schematic representation of a cell layer in a multilayer architecture (4A), and an optical microscope photograph of a layer of human erythrocytes incorporated within a multilayer film (4B);

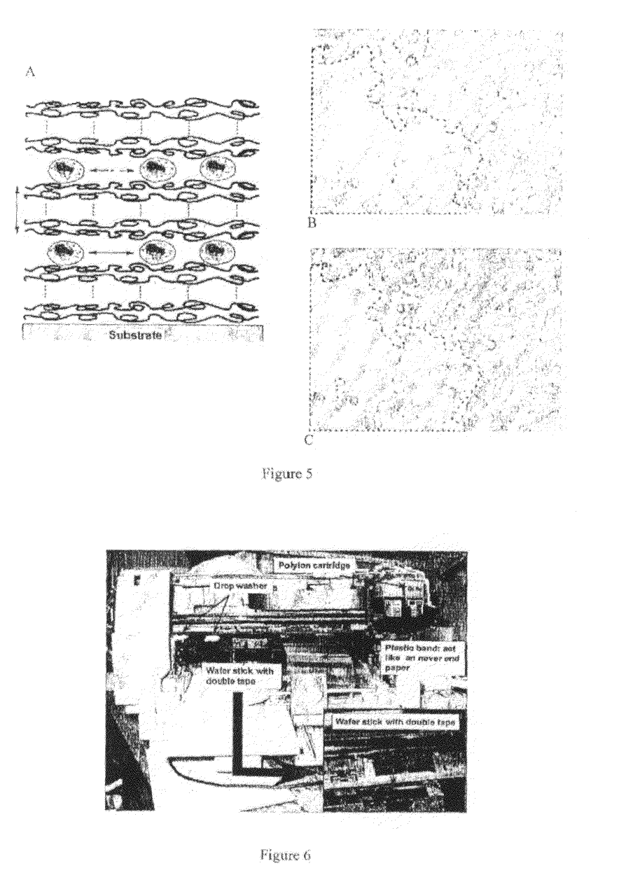

FIGS. 5A-5C show a schematic representation of two cell layers in a multilayer architecture (5A), and two optical microscope photographs of two layers of human erythrocytes incorporated within a multilayer film (5B and 5C);

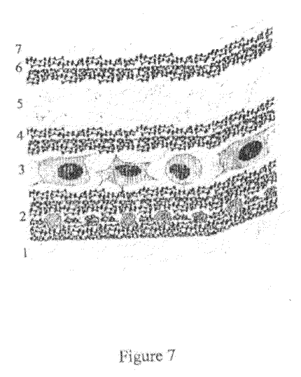

FIG. 6 shows a photograph of the HP 690C ink-jet printer with magnification of the substrate at the print zone;

FIG. 7 shows a cross-section of the strategy according to the invention for spray deposition of the various cartilage components.

### EXAMPLE

### Manufacture of Functional Biohybrid Objects

FIG. 3 shows the construction of a biohybrid object by 3D layer-by-layer-deposition using an ink-jet printer. In this schematic representation, the print head includes three nozzles for distributing liquids, containing, for example, living cells, polyanions and polycations, respectively. For the preparation of complex biomaterials, print heads are used which include the required number of nozzles for the various depositions to be carried out. The liquids containing the desired components are deposited in quantities ranging from picolitres to microlitres so as to form successive layers of the component elements of the biomaterial and of the extracellular matrix. This method involves, for example; interactions between opposite charges (electrostatic LbL), other non-covalent interactions (e.g. hydrogen bonds), and covalent interactions (covalent LbL) between the materials which make up the layers. Successive deposits are made in such a way as to control the thickness and the 2D and 3D structure of each layer.

In order to form the hydrogel-type artificial matrix, layers of polysaccharides or of exponentially growing biodegradable peptides are deposited, allowing the formation of thick layers with a hydrogel character, but with a loose structure, whilst carrying out only a small number of cycles.

By combining linearly growing layers with exponentially growing layers, films with several compartments may be manufactured, if desired.

Protocol for the Preparation of Multilayers which Contain Erythrocytes on Glass:

Erythrocytes were selected because these cells are non-functional and difficult to immobilise. The immobilisation of these cells therefore poses a real challenge, and their ability to pass through small-size vascular structures make them perfectly suitable for the micro-shear induced by spraying.

The lack of disruption of erythrocytes in the presence of PDDAC (Poly Diallyl Dimethyl Ammonium Chloride) in contrast to PLL (Poly-L-Lysine) or PAH (Poly Allylamine Hydrochloride) lead to the use of this polymer for direct contact with the blood material (the layer before and after deposition of non-encapsulated erythrocytes in the case of deposition onto a surface, as well as the layer for encapsulating erythrocytes).

### A) Activation of the Surface:

The substrates (circular glass slides) were previously cleaned with ethanol, and if necessary with acetone, then dried under a stream of nitrogen in order to give a clean surface. The slides were then activated by sequential immersion in a solution of HCl/MeOH (50/50 v/v) and in a concentrated H₂SO₄ solution for at least 4 hours each. For these various operations, a Teflon rack made earlier at ICS was used, which holds 6 slides vertically.

### B) Preparation of Polyelectrolyte Solutions:

The polyelectrolyte solutions were prepared at 0.1% (w/v) in Tris buffer (25 mM) containing NaCl (0.13-0.15 M) and with pH adjusted to 7.3 (±0.1). The polyelectrolytes used for the construction of the multilayers were: PEI (Poly Ethylene Imine), alginate, PLL, PSS (sodium Poly Styrene Sulphonate), PAH, and PDDAC.

### C) Preparation of Blood Aliquots:

A sample of human whole blood was centrifuged and rinsed at least once with about 12 mL of Tris buffer (see section B). A 300 μL aliquot of this preparation was taken and diluted to qsp 1 mL in Tris buffer before deposition onto the surfaces previously activated and treated using multilayers terminated with PDDAC.

### D) Construction of the Mixed Multilayer:

The activated slides were rinsed with ultrapure water before the deposition of polyelectrolyte layers.

### *Deposition of a Polyelectrolyte Layer:

The slides were immersed in about 15 mL of a polyelectrolyte solution as described in B) for about 15 minutes. They were then rinsed twice with 15 mL of buffer for 2 minutes each, with manual agitation of the rack for about 1 minute. The process was repeated using a solution of polyelectrolyte of the opposite charge, as many times as necessary to achieve the desired construction (see below).

### *Deposition of a Cell Layer:

A sample was taken from the diluted aliquot using a Pasteur pipette and deposited onto the treated slides (PDDAC-terminated) so that the drop covered the entire surface of the slide. After 20-minutes contact time, the slide was rinsed in the same manner as for the polyelectrolyte layers. Alternatively, the deposition of erythrocytes may also be carried out using spraying.

The construction of a multilayer always starts with PEI. The following layer contains (PSS/PDDAC)ₙ either directly, or after prior adsorption of (PSS/PAH)ₙ with FITC-coupled PAH (fluorophore) in order to reveal the fluorescence via confocal microscopy. The layers directly covering the erythrocytes are made up either of (PDDAC/PSS)ₙ or (PDDAC/PSS)ₙ, then covered with PAH/PSS with fluorescent PAH to reveal the coating via confocal microscopy or the fluorescence. In the case of erythrocytes encapsulated (14, 15) in PDDAC, they are incorporated within the multilayer between two layers of polyanions.

In the case of several layers of erythrocytes being deposited, erythrocytes may either be directly deposited onto the multilayer (a small number of polyelectrolyte layers is sufficient), or a hydrogel-type multilayer (exponential growth) may also be constructed between the two cell layers in order to ensure the spacing (here, (PLL/Alg)ₙ in which n is from 8 to 20).

FIG. 4 shows an optical microscopy image of human erythrocytes incorporated within a multilayer film (magnification: ×400).

FIGS. 5B and 5C show two optical microscopy images in which 2 layers of human erythrocytes have been incorporated within a multilayer. These two images differ only in that the focus is on each cell layer.

Control over the Local Environment:

In order to exercise control over the local environment around each cell, each cell was functionalised on its surface using LbL coating with biofunctional macromolecules.

Alternatively, active agents such as RGD (a tripeptide which facilitates cell adhesion) or α-MSH (an antiinflammatory agent) were attached to polyelectrolytes such as poly-L-lysine (16-19), which resulted in polymers which may easily be characterised in solution and deposited onto various surfaces using the layer-by-layer deposition method.

FIG. 6 shows the HP 690C printer used to print “ICP Strasbourg” with a multilayer of polyelectrolytes on a silicon substrate.

### Protocol for Printing a Polyelectrolyte Film Using a HP 690C Printer:

### A) Preparation of Solutions:

PSS and PAH were used at a concentration of 1 mg/mL in Milli-Q aqueous solution. No salt was used.

### B) Preparation of a Printer Cartridge:

First of all the single black-ink cartridge used was emptied by punching a hole in the membrane on the upper face of the cartridge. Once empty, the cartridge was cleaned with tap water until there was no more ink inside the cartridge and no more ink flowed out from it. Finally, the cartridge was rinsed thoroughly with a solution of Mill-Q water, then dried with nitrogen. Polyelectrolyte solutions were introduced using a syringe through the upper face.

### C) Preparation of the Printer:

The printer used, a HP 690C model in which the black cartridge is separate from the colour cartridge, can produce prints from a single ink cartridge—the black cartridge in these experiments.

The substrate was adhered to the black plastic bar of the printer where printing was performed. The print zone was pre-calibrated using the Canevas software, by printing first of all on a blank page and then onto a tape adhered to the plastic bar.

### D) Printing a Multilayer Film:

To print a multilayer film onto a silicon substrate, the same pattern (“ICS Strasbourg”) was printed alternately using a cartridge containing PSS and a cartridge containing PAH, respectively. Since the cartridge contains no salt, printing can be carried out without rinsing.

Note: If it is desired to print using solutions which contain salt, the surface must be rinsed after each layer is deposited since the salt crystals rapidly appear at the surface. For this, the substrate must be removed and re-adhered in the same place.

Protocol for Immobilisation of Cells by Gelification of Alginate Sprayed onto a Calcium-Rich Surface:

The sodium alginate was solubilised at 0.6% (w/v) in a Krebs-Ringer-Hepes buffer, made up of 25 mM Hepes, 90 mM NaCl, 4.7 mM KCl, 1.2 mM HK₂PO₄, and 1.2 mM MgSO₄.7H₂O, with pH adjusted to 73 using a 1M NaOH solution. The cells were then dispersed in the alginate solution. The suspension thus obtained was sprayed at an approximate pressure of 1.3 atm onto a Petri dish which has previously been treated with a (PLL/Alg)₂/Ca multilayer. Finally, the material containing the cells was rinsed by sequential immersion in two Hepes-buffer baths. An additional deposition of layers of polyelectrolytes onto the alginate cells may be carried out.

### Protocol for Encapsulation of Cells Using Polyelectrolytes:

Individual encapsulation of cells was achieved by successive dispersion of the cells in a 0.3% (w/v) alginate solution in Hepes buffer, and in a 0.1% (w/v) PLL solution in Hepes buffer for 15 minutes. Between each layer, the suspension was centrifuged at 2,000 rpm for 5 minutes, rinsed with Hepes buffer, and centrifuged again.

### Three-Dimensional Reconstruction of Cartilage by Sequential Spraying of Cells and Matrix Constituents:

Cartilage matrix components were deposited by simultaneous and/or alternating spraying as three cell-matrix sub-units with different structures and natures, namely:
(i) a (hyaluronic acid (HA)/PLL) film as active compartment,
(ii) a calcium alginate gel containing the cells,
(iii) a mixture of collagen and hydroxyapatite.

The alginate gels and the films which are functionalised, e.g. using growth factors, act as supports in the process of cell differentiation and cartilage regeneration, whereas the mixture of collagen and hydroxyapatite facilitate the anchorage of the biomaterial onto the subchondral bone.

This construction is represented in FIG. 7, which shows in cross-section the strategy followed for deposition by spraying of the various cartilage constituents used:
(1) a layer of collagen and apatite as an osteochondral matrix,
(2) a multilayer film (e.g. the HA/PLL system) as a compartment which is functionalised by growth factors of interest such as BMP-2 and TGF-β,
(3) an alginate layer containing stem cells, to be differentiated into chondrocytes, expressing type-X collagen,
(4) a multilayer film (HA/PLL) as active compartment,
(5) an alginate layer containing stem cells, to be differentiated into chondrocytes, expressing cartilage-specific type-IIB collagen,
(6) a multilayer film (HA/PLL) as active compartment,
(7) an alginate layer containing stem cells, to be differentiated into chondrocytes, expressing type-I and II collagen.

The two reagents for forming the calcium alginate gel were CaCl₂ and sodium alginate, which is a natural polymer made up of two monosaccharide units, D-mannuronic acid (M) and L-guluronic acid (G). The proportions of M and G units vary from one species to another.

In the experiments reported in these examples, the sodium alginate used comes from the alga Macrocystis pyrifera (Sigma-Aldrich). This sodium alginate includes a large proportion of M units and the M/G ratio is about 1.6. The gel is obtained by simultaneous spraying of a solution of Ca²⁺ ions at a concentration of 0.125 M and of a sodium alginate solution with a Ca/alginate molar ratio of 1/1. For spraying of the cells, the cells were suspended in the sodium alginate solution.

The viability of the cells sprayed in calcium alginate gels has been monitored using the MTT method, thus confirming that spraying of cells in gels does not result in significant cell death.

Confocal microscopic observation of gels containing fibroblasts has shown that the cells actually remain between two layers of gel and retain their phenotype.

### BIBLIOGRAPHIC REFERENCES

1. Langer R., Vacanti J. P., Tissue engineering, Science, 1993, 260, 920-926.
2. Patents from Yamato M. et al. based on the concept of “Cell Sheet” used in various tissular engineering applications: EP 1600177—2005, EP 1598417—2005, WO 2005103233—2005, JP 2005130838—2005, US 2004009566—2004, US 2004028657—2004, JP 2005000608—2005, JP 2004261533—2004, JP 2004261532—2004, US 2003036196—2003, JP 2003038170—2003, EP 1264877—2002, JP 5168470—1993.
3. Boland T., Cris Wilson JR. W., Xu T., Ink-jet printing of viable cells, U.S. Pat. No. 237,822—2004.
4. Barron J. A., Spargo B. J., Ringeisen B. R., Biological laser printing of three-dimensional cellular structures, Appl. Phys. A., 2004, 79, 1027-1030.
5. Decher G., Hong J.-D., One- or multi-layered layer elements applied to supports and their production, U.S. Pat. No. 520,811—1993.
6. Decher G., Fuzzy Nanoassemblies: Toward Layered Polymeric Multicomposites, Science, 1997, 277, 1232-1237.
7. Multilayer Thin Films: Sequential Assembly of Nanocomposite Materials; Decher, G. and Schlenoff, J. B., eds., Wiley-VCH: Weinheim, 2003; 524 pages.
8. Voegel J.-C., Decher G., Schaaf P., Multicouches de polyélectrolytes dans le domaine des biotechnologies, L'Actualité Chimique, November-December 2003, 28-36.
9. Schlenoff J. B., Dubas S. T., Farhat T., Sprayed polyelectrolyte multilayers, Langmuir, 2000, 16 (26), 9968-9969.
10. Izquierdo, S. S., Ono, J.-C., Voegel, P., Schaaf G., Decher, Dipping versus spraying: exploring the deposition conditions for speeding up Layer-by-Layer assembly, Langmuir, 2005, 21, 7558-7567.
11. Nahmias Y., Arneja A., Tower T. T., Renn M. J., Odde D. J., Cell Patterning on Biological Gels via Cell Spraying through a Mask, Tissue Eng., 2005, 11 (5-6), 701-708.
12. Neu B., Baumler H., Donath E., Moya S., Sukhobukov G., Möhwald H., Caruso F., Polyelectrolyte coverings on biological templates, U.S. Pat. No. 6,699,501—2004.
13. Diaspro A., Silvano D., Krol S., Cavalieri O., Gliozzi A., Single living cell encapsulation in nano-organized polyelectrolyte shells, Langmuir, 2002, 18, 5047-5050.
14. Frank Caruso, Hollow Capsule Processing through Colloidal Templating and Self-Assembly, Chem. Eur. J., 2000, 6 (3), 413-419.
15. Neu B., Voigt A., Mitlöhner R, Leporatti S., Gao C. Y., Donath R., Kiesewetter H., Möhwald H., Meiselman H. J., Bäumler H., Biological cells as templates for hollow microcapsules, Microencapsulation, 2001, 18 (3), 385-395.
16. Chluba J., Voegel J.-C., Decher G., Erbacher P., Schaaf P., Ogier J., Peptide hormone covalently bound to polyelectrolytes and embedded into multilayer architectures conserving full biological activity, Biomacromolecules, 2001, 2 (3), 800-805.
17. Benkirane-Jessel N., Lavalle P., Meyer F., Audouin F., Frisch B., Schaaf P., Ogier J., Decher G., Voegel J.-C., Control of monocyte morphology on and response to model surfaces for implants equipped with anti-inflammatory agents, Adv. Mater., 2004, 16 (17), 1507-1511.
18. Schultz P., Vautier D., Richert L., Jessel N., Haikel Y., Schaaf P., Voegel J.-C., Ogier J., Debry Polyelectrolyte multilayers functionalized by a synthetic analogue of an anti-inflammatory peptide, alpha-MSH, for coating a tracheal prosthesis, Biomaterials, 2005, 26 (15), 2621-2630.
19. Picart C., Elkaim R., Richert L., Audoin T., Arntz Y., Cardoso M. D., Schaaf P., Voegel J.-C., Frisch B., Primary cell adhesion on RGD-functionalized and covalently crosslinked thin polyelectrolyte multilayer films, Adv. Funct. Mater., 2005, 15 (1), 83-94.

1. A method of construction of a artificial functional living biomaterial, characterised by the layer-by-layer assembly (3D) of a matrix and of 2D layers of functional living cells by controlling their interactions and 3D structure depending on the final desired organisation and form of the biomaterial, in which each layer has its own pattern which is suited to the neighbouring layers and to the functionality of the entire artificial biomaterials.

2. The method according to claim 1, characterised by 2D cell layers formed either in an homogenous manner (non-structured layers) by soaking, spraying or using a method which gives a thin hydrogel layer (with nanometre to micrometre-scale thickness) during the immobilisation period, or in a heterogeneous manner (layers structured in specific patterns) by cell-by-cell spotting using a printer or a robot system.

3. The method according to claim 1, characterised by each individual cell being provided, at a local level, with the elements required for it to survive, such as nutrients, oxygen-transportation or scavenging agents, growth factors, cell-differentiation factors, antioxidants, cell-adhesion promoters, antiinflammatory agents, antibacterial agents, antiviral agents, angiogenesis factors or inhibitors, immunosuppressive agents, co-factors, vitamins, DNAs, gene-transfection agents, or any factor which stimulates or suppresses cellular, biological or therapeutic activity.

4. The method according to claim 1, for the construction of a simple biomaterial, characterised in that a single layer of cells is deposited, in which these cells, immobilised using the layer-by-layer method, are of the same type or of at least two different cell types.

5. The method according to claim 1, for the construction of a complex biomaterial, characterised in that several successive alternating layers of polymers and cells of different types, depending on the desired architecture, are deposited.

6. The method according to claim 1, characterised in that the deposited cells are cells which form the final surface of the organ (skin), endothelial cells, smooth-muscle cells, connective-tissue cells, cells which make up blood vessels, cells which make up tissues or organs, and stem cells whose differentiation into functional cells is induced by differentiation factors, excluding embryonic stem cells of human origin.

7. The method according to claim 1, characterised in that, in order to achieve control of the cell-matrix interactions in space, naked cells or cells individually coated with multilayers of polyelectrolytes, or cells immobilised in a thin layer of nascent hydrogel (with nanometre to micrometre-scale thickness) are used.

8. The method according to claim 1, characterised in that living cells encapsulated in a coating having a nanometre-scale thickness are used, in which this coating includes functional macromolecules suitable for coating of cells.

9. The method according to claim 1, characterised in that the extracellular matrix, made up of biotolerant or bio-inert macromolecules, or if necessary biodegradable or bioactive molecules, is constructed layer by layer using the LbL method, from molecules, macromolecules or colloidal compounds, e.g. hydrogel in the form of nanoparticles or vesicles, which interact by covalent and/or non-covalent interactions, or by gelification of two or more compounds, such as, for example, sodium alginate and calcium.

10. The method according to claim 1, characterised in that, during construction, the molecules are incorporated either into the multilayer matrix or on either side of the matrix.

11. The method according to claim 1, characterised in that immobilisation of cells onto the matrix is achieved:
by deposition of naked cells or cells which are individually encapsulated using multilayers of electrolytes, onto a substrate which has been modified beforehand using a multilayer treatment whose surface is of opposite charge;
by incorporation of cells into a hydrogel matrix which is forming on the surface and which is made up, for example, of calcium alginate, whose thickness depends on the thickness of the multilayer reservoir which controls the concentration of one of the compounds necessary for gelification;
by inclusion of cells into a hydrogel matrix which is forming on the surface and which is made up, for example, of calcium alginate, but without a multilayer reservoir being involved.

12. Biomaterials as obtained by the method defined in claim 1, as new products.

13. An application of the biomaterials according to claim 12, in the biomedical and nanobiotechnology fields.

14. The application according to claim 13, as interface or membrane reactors in thin layers for the biomanufacture of molecules of interest, by living cells.

15. The application according to claim 13, as an artificial organ which is either completely biohybrid, or as a synthetic device coated with a biohybrid layer.

16. The application according to claim 13, for tissue repair, in particular for the repair of cartilage.
